(19) United States
(12) Patent Application Publication
(10) Pub. No.: US 2013/0150882 A1
(43) Pub. Date: Jun. 13, 2013

(54) APPARATUS AND METHOD FOR DIVERTING EMBOLIC MATERIAL

(71) Applicants: Michael S. Williams, Santa Rosa, CA (US); Grant Williams, Larkspur, CA (US); Richard A. Glenn, Santa Rosa, CA (US)

(72) Inventors: Michael S. Williams, Santa Rosa, CA (US); Grant Williams, Larkspur, CA (US); Richard A. Glenn, Santa Rosa, CA (US)

(21) Appl. No.: 13/536,954

(22) Filed: Jun. 28, 2012

Related U.S. Application Data
(60) Provisional application No. 61/501,771, filed on Jun. 28, 2011.

(51) Int. Cl. A61F 2/01

(52) U.S. Cl. CPC A61F 2/01
USPC 606/200

(57) ABSTRACT

An embolic deflection device includes an anchor engageable with a lumen of a branch vessel extending from an aortic arch and a deflection device, the deflection device having a first end coupled to the anchor and an elongate member extending from the deflection device. A method of deflecting embolic material away from a branch vessel extending from an aortic arch includes providing a deflection device comprising a anchor and an elongate member extending from the anchor. The anchor is engaged with a lumen of the branch vessel and positioned such that the elongate member extends within the aortic arch in proximity to ostia of at least two branch vessels. Embolic material flowing from the ascending aorta to the aortic arch is diverted by the elongate member away from the branch vessels and into the descending aorta.

This application claims the benefit of U.S. Provisional Application No. 61/501,771, filed 28 Jun. 2011, which is incorporated herein by reference.

### BACKGROUND

The human aorta includes an ascending portion and a descending portion, and one or more arch vessels communicating with the aorta for directing blood flow to the brain of the patient. U.S. Patent Application 2008/0140110 (the “110 application”, which is incorporated herein by reference), entitled Implant, Systems and Methods for Physically Diverting Material in Blood Flow Away from the Head, describes a device for preventing stroke due to embolic material in the bloodstream of a patient. The device includes a physical deflector element configured for at least partial placement in the aorta of the patient and a mounting structure coupled to the physical deflector element. The mounting structure is configured to engage at least one of the aorta or an arch vessel communicating with the aorta. The physical deflector element is constructed and arranged to direct blood flow in the aorta in a manner that directs embolic material in the blood flow past the one or more arch vessels and into the descending portion of the aorta.

The present application describes alternatives to the devices disclosed in the '110 application.

### BRIEF DESCRIPTION OF THE DRAWINGS

FIGS. 1 and 2 schematically show a first embodiment of a thromboembolic diverter positioned in an aorta;

FIG. 3 is a perspective view of the diverter of FIG. 1;

FIG. 4 is a side elevation view of the diverter of FIG. 1;

FIG. 5 is a perspective view of an alternate thromboembolic diverter;

FIG. 6 schematically shows the diverter of FIG. 5 positioned in an aorta;

FIGS. 7A-7B are a sequence of drawings schematically illustrating removal of the diverter of FIG. 1 from the aorta;

FIGS. 8A through 10 B show alternative diverter designs.

### DETAILED DESCRIPTION

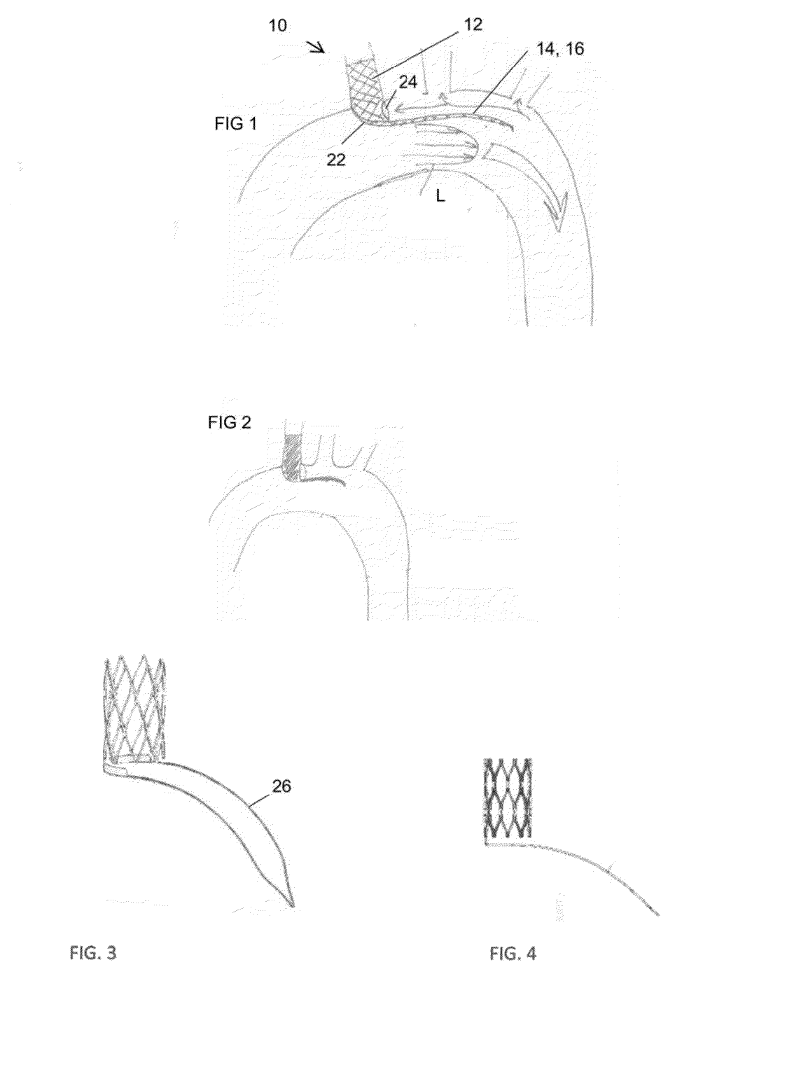

FIG. 1 shows a first embodiment of a thromboembolic diverter 10 positioned within the aortic arch. Diverter 10 includes an anchor 12 and a deflector 14.

The anchor 12 is positioned within an arch vessel such as the brachiocephalic artery as shown. Anchor 12 may be a tubular member that is deliverable into the arch vessel in a radially compressed state, and is expandable into contact with the arch vessel walls once in the desired position. Expansion may be carried out using a separate device positioned within the anchor, such as a balloon, or the anchor may be self-expandable.

The deflector 14 is coupled to the portion of the anchor 12 that is closest to the aorta. The deflector includes an elongate member 16 that extends within the aortic arch, below the ostia of one or more of the arch vessels. In the illustrated embodiment, the member 16 extends beneath all three of the ostia, although in other embodiments it extends beneath just one or two of the ostia.

Member 16 includes a first surface facing the ostia, and a second surface that is opposite from the first surface. The deflector 14 is preferably contoured such that the first and second surfaces curve generally in parallel to the contour of the aortic arch such that a channel of generally uniform cross-section is formed along the second surface (between the first surface and the opposing walls of the aortic arch), and another channel (preferably also of generally uniform cross-section) is formed along the first surface—between the first surface and the opposing walls of the aortic arch. The long edges of the member 16 may extend laterally into contact with the walls of the aorta, or the member 16 may be narrower than the corresponding diameter of the aorta so that some blood can pass between the long edges and the adjacent walls of the aorta.

The diverter 10 includes a barrier region 22 disposed within the aortic arch, upstream of the ostia of the arch vessel within which the anchor 12 is positioned. In the illustrated embodiment, one end of the anchor 12 extends into the aorta and its upstream-facing portion forms this surface. In other embodiments, the barrier region 22 may be part of the deflector 14 or a transition section of the diverter 10 in the region where the deflector and anchor 12 are coupled together. Downstream of the barrier is an entrance 24 for passage of blood into the ostium within which the anchor 12 is positioned. The entrance may be an orifice or an opening in the diverter 10.

The diverter 10 may be constructed in a variety of ways. For example, the anchor 12 may have a stent-like construction formed using a tubular shape memory device constructed of nitinol, shape memory polymer, or other suitable materials. The device (anchor and/or deflector) may be of a woven construction or formed from laser cut tubing. The anchor may include features that give some structural stability to cause the anchor to radially support the device against a vessel wall. For example, a mesh, band or other framework formed of shape memory (e.g. nickel titanium alloy, nitinol or shape memory polymer) elements or stainless steel, Eligoy, or MP35N wires or structures may be used. The anchor may include a smooth polymeric barrier that is both anti-proliferative and anti-thrombogenic and that thereby prevents endothelial growth and thrombus formation on the anchor. Examples of materials for the polymeric barrier include, but are not limited to ePTFE, or other fluoropolymers, silicone, non-woven nylon, or biomimetic materials. The deflector 14 may include an outer frame defining the edges of the deflector, with the elongate member 16 disposed within the outer frame. The elongate member 16 may be formed of a variety of materials, such as silicone, ePTFE, polyurethane, kapton, or dacron or other woven material. The elongate member preferably includes a surface or coating of anti-thrombogenic material such as heparin.

As illustrated in FIG. 1, when embolic particles are carried by blood flow into the aorta, the elongate member deflects the particles away from the branch vessels, and creates or enhances a laminar flow L of blood within the aortic arch which can carry particles away from the branch vessels and downstream into the descending aorta, and/or facilitate acceleration of particles away from the blood flow entrance towards, the descending aorta. In either case, blood may flow around the elongate member as shown, and into the branch vessels.

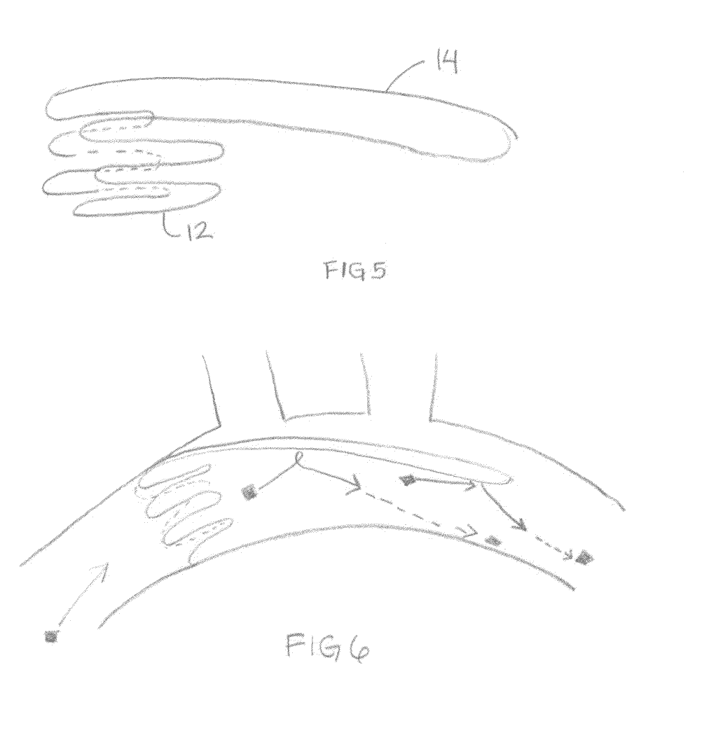

FIGS. 3 and 4 show one variation of the FIG. 1 diverter, in which the anchor 12 and deflector 14 are integral pieces formed from a single laser cut tube. In this embodiment, the portion of the tube that will become the deflector is cut to form an elongate support 26, which preferably has a tapered distal- or upstream-end (i.e. the end furthest from the anchor). The support 26 may take the form of a frame defining an open area, or it may have other configurations. The support 26 is shape set to give the frame the curvature shown. The open area(s) surrounded by the frame or otherwise defined by the support are filled or covered by a material such as polyurethane, silicone, or other material that will prevent passage of emboli through the frame. In one embodiment, the covering may be applied by dipping the frame in the material that is to form the elongate member.

FIGS. 5 and 6 show an alternative to the FIG. 3 diverter, in which the anchor 12 and deflector 14 are arranged such that the anchor 12 is engaged with the walls of the aorta (in this embodiment upstream of the brachiocephalic artery). As with the prior embodiment, this embodiment may be formed from a single tube, laser cut to form the anchor and a frame for the deflector, then shape-set if needed to shape the deflector. A covering is then applied to the support as discussed above. FIG. 6 illustrates positioning of the diverter and use of the diverter to deflect particles away from the ostia to the carotid and brachiocephalic arteries, causing the particles to continue to move downstream rather than passing into the head vessels.

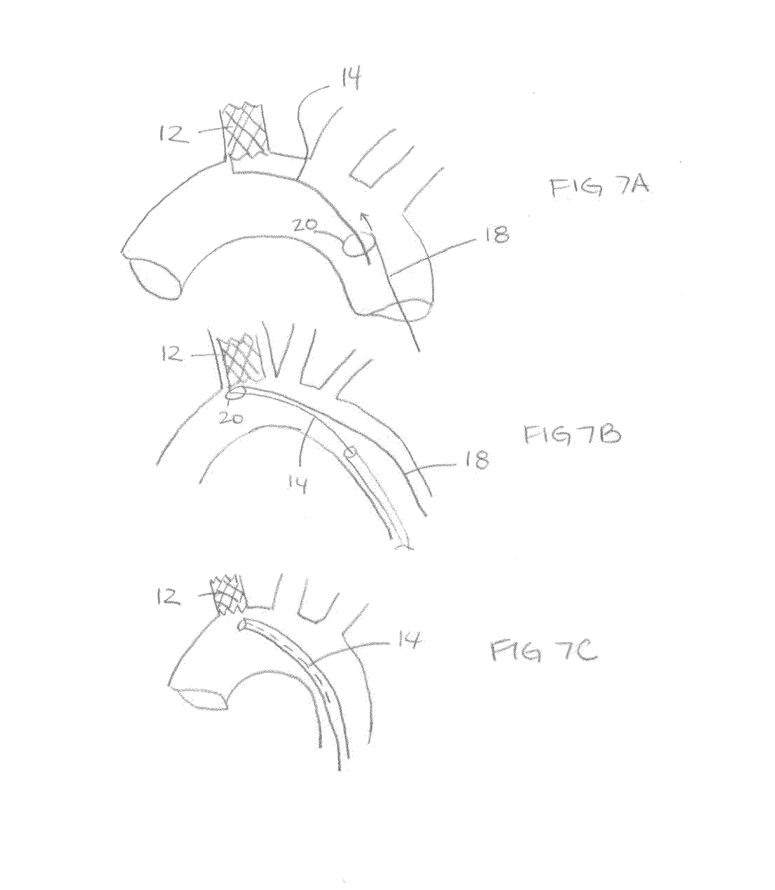

FIGS. 7A-7C are a sequence of views illustrating use of a cutting device to detach the deflector from the anchor in an embodiment such as those shown in FIGS. 1-6. A cutting tool 18 having a snare 20 on it is advanced into the aorta (such as via the descending aorta as shown in FIG. 7A) and positioned with the snare loop over the deflector 14. The snare loop is advanced to the base of the anchor as shown. Referring to FIG. 7B, a retrieval catheter 22 is advanced over the deflector 14, preferably up to the snare loop. The snare loop is used to cut the deflector from the anchor 12. The retrieval catheter 22 is withdrawn (with the severed deflector within it as shown in FIG. 7C), leaving only the anchor 12 behind. This method of removing the deflector minimizes trauma to the vessel within which the anchor is seated by leaving the anchor in place. In other extraction methods, both the anchor and the deflector may be removed as a single piece or as separate pieces.

FIG. 5 and related figures of the '110 application illustrate embodiments of individual tubular deflector elements that may be inserted and mounted within the entrances to the arch vessels (e.g. brachiocephalic, carotid) extending from the aortic arch. Each of the disclosed tubular elements includes a blood flow entrance and a blood flow exit end. The exit end of each tube extends within the respective arch vessel, while the entrance end extends within the aorta. A bend in the tube locates the entrance end a suitable or desired distance downstream in the aorta such that particles will tend to be deflected by the tubular member and continue within the blood flow as opposed to reversing direction and entering the entrance end of one of the tubes.

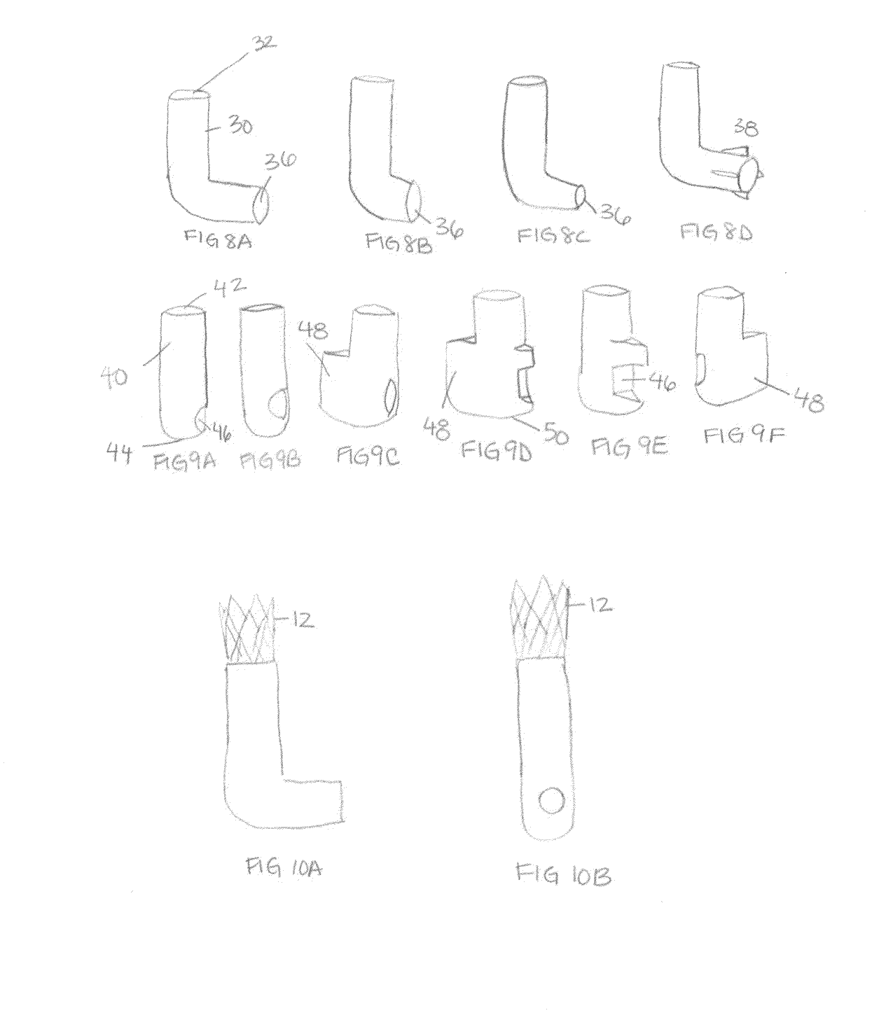

FIGS. 8A through 8D illustrate alternative shapes to the tubes shown in the '110 application, which may be made of silicone or other polymeric material anchored within the arch vessel. In these embodiments, the deflector element includes a first portion 30 of tubing (shown as generally vertical in the drawings) that defines the blood flow exit 32 (which is anchored within an arch vessel) and a second portion 34 of tubing extending laterally from the first portion (and shown as generally horizontal in the drawings) which defines the blood flow entrance 36 (which is disposed in the aortic arch). FIGS. 8A and 8B illustrate embodiments in which, the second portion 34 has a length that is equal in length or longer than the length of the first portion 30 (FIG. 8A), or is shorter than the length of first portion 30 (FIG. 8B). In FIG. 8C, the second portion 34 tapers to a narrower diameter at its distal end. In FIG. 8D, the second portion 34 includes a plurality (four shown) of baffles 38 extending radially from the outer surface of the tubing.

FIGS. 9A through 9F show alternatives to the FIG. 8 embodiments. Each of these embodiments includes a tubular device 40 made of silicone or other polymeric material anchored within the arch vessel. At one end of the tube is an exit opening 42 disposed within the arch vessel. The opposite end 44 of the tube is closed. A blood flow entrance 46 in the tubular device comprises an opening formed in a downstream wall of the tube. In these embodiments, the exit opening 42 is disposed within the arch vessel, and the entrance opening 46 is disposed in a downstream-facing direction within the aortic arch. In the FIG. 9A embodiment, the entrance 46 is adjacent to the lower end 44 of the tube, whereas in the FIG. 9B embodiment the entrance 46 is positioned further up the tubular wall, closer to the exit opening 42. The FIG. 9C-9F embodiments incorporate elements designed to encourage laminar flow of blood within the aortic arch and/or to facilitate acceleration of particles away from the blood flow entrance. In FIGS. 9C, 9D and 9F, an upstream element 48 which may be a wedge shaped element positioned with the narrow end of the wedge extending upstream. FIGS. 9D and 9E show use of a pair of wedge-shaped downstream elements 50 positioned on opposite sides of the entrance opening 46, with the narrow end of each wedge extending downstream.

FIGS. 10A and 10B are similar to the FIG. 8B and FIG. 9B embodiments, respectively, but comprises an expandable stent-like anchor 12 disposed within the blood flow exit opening of the tubular device. The anchor and tube may be implanted separately or as a singular piece. For example, the device is positioned with the blood flow exit opening within a branch vessel, in proximity to the ostia at the aortic arch, and with the entrance opening disposed within the aortic arch. Once the device is positioned, the stent-like anchor 12 is delivered through the lumen of the device (e.g. carried in a compressed position within a deployment sheath or catheter) and expanded, such that expansion of the stent-like anchor within the arch vessel causes the upper end of the tubular device to be engaged between the expanded anchor and the surrounding vessel wall.

We claim:

1. An embolic deflection device, comprising:
an anchor engageable with a lumen of a branch vessel extending from an aortic arch; and
a deflection device, the deflection device having a first end coupled to the anchor and an elongate member extending from the deflection device.

2. A method of deflecting embolic material away from a branch vessel extending from an aortic arch, comprising:
providing a deflection device comprising a anchor and an elongate member extending from the anchor;
engaging the anchor with a lumen of the branch vessel and positioning the elongate member to extend within the aortic arch in proximity to ostia of at least two branch vessels, wherein embolic material flowing from the ascending aorta to the aortic arch is diverted by the elongate member away from the branch vessels and into the descending aorta.
